(19) United States
(12) Patent Application Publication
(10) Pub. No.: US 2013/0204145 A1
(43) Pub. Date: Aug. 8, 2013

(54) SYSTEM AND METHOD FOR MANAGING DEVICES AND DATA IN A MEDICAL ENVIRONMENT

(71) Applicant: Shahid N. Shah, Silver Spring, MD (US)

(72) Inventor: Shahid N. Shah, Silver Spring, MD (US)

(73) Assignee: Netspective Communications LLC, Silver Spring, MD (US)

(21) Appl. No.: 13/756,445

(22) Filed: Jan. 31, 2013

Related U.S. Application Data
(60) Provisional application No. 61/594,224, filed on Feb. 2, 2012.

(51) Int. Cl. H04L 29/08; A61B 5/0205

(52) U.S. Cl. CPC H04L 67/02; A61B 5/0205
USPC 600/484; 709/201

(57) ABSTRACT

A system and method for facilitating coordinated functioning of medical devices over a network. The system includes a communication circuit to receive an input indicative of an action to be performed by a first medical device from a processing unit coupled to a social health record data bank. The communication circuit is configured to send an instruction to the first medical device to initiate the action by the first medical device in association with information of a patient associated with the first medical device stored in and retrieved from the social health record data bank. The system further includes a control circuit configured to monitor the action performed by the first medical device and instruct the first medical device to pause performing the action for a defined period of time based on an instruction from the processing unit indicative of an action to be performed by a second medical device.

### CROSS-REFERENCE TO RELATED APPLICATIONS

This application claims the benefit of U.S. Provisional Application No. 61/594,224, filed on Feb. 2, 2012, the complete disclosure of which, in its entirety, is hereby incorporated by reference.

### BACKGROUND

1. Technical Field

The embodiments herein generally relate to data and devices management, and more particularly, to healthcare data and devices management and methods.

2. Description of the Related Art

Hospitals, caretakers, nursing centers or homes, medical offices, medical centers, or other sources of medical care and entities generally keep medical and demographic or other such records of their patients. These records may include a variety of information such as demographic information of their patients, medical history, diagnostic and pathology reports of their patients, medical reports or prescriptions, or other such information. This information can be used for a variety of purposes by these sources of medical care. A few examples of them are, without limitations, tracking of the patients and their records, billing, historical assessments, integrating with medical devices, remote care, future care taking, telemedicine, proper ongoing medical or health assessment or treatment, or any other purpose.

One way to collate and store the medical data is with the use of an electronic health record data bank (EHRDB). These records from various entities can be electronically maintained such as by the electronic health record data bank (EHRDB) in a central system accessible by the entities. The EHRDB may store medical data of the entities and devices and retrieve the data of the respective entities as and when requested by them.

There is a need for an improved system and a method that provides a facility for the devices and other entities to interact with the EHRDB and that may allow coordinated functioning of the devices with the use of the EHRDB.

### SUMMARY

An embodiment herein provides a system for facilitating coordinated functioning of medical devices over a network. The system includes a communication circuit configured to receive an input indicative of an action to be performed by a first medical device from among a plurality of networked medical devices, the input being received from a processing unit coupled to a social health record data bank. The communication circuit is further configured to send an instruction to the first medical device to initiate the action by the first medical device in association with information of a patient associated with the first medical device stored in and retrieved from the social health record data bank. The system further includes a control circuit configured to monitor the action performed by the first medical device and instruct the first medical device to pause performing the action for a defined period of time based on an instruction from the processing unit indicative of an action to be performed by a second medical device.

Another embodiment herein provides a method for facilitating coordinated functioning of a plurality of medical devices over a network. The method includes receiving an input indicative of a first action to be performed by a first medical device from among a plurality of networked medical devices, the input being received from a processing unit coupled to a social health record data bank. The method includes sending an instruction to the first medical device to initiate the first action by the first medical device in association with information of a patient associated with the first medical device stored in and retrieved from the social health record data bank. The method includes monitoring the first action performed by the first medical device in accordance with the instruction. The method includes instructing the first medical device to pause performing the action for a defined period of time based on an instruction from the processing unit indicative of a second action to be performed by a second medical device.

Another embodiment herein provides a program storage device readable by computer, and comprising a program of instructions executable by said computer to perform a method for facilitating coordinated functioning of a plurality of medical devices over a network. The method includes receiving an input indicative of a first action to be performed by a first medical device from among a plurality of networked medical devices, the input being received from a processing unit coupled to a social health record data bank. The method includes sending an instruction to the first medical device to initiate the first action by the first medical device in association with information of a patient associated with the first medical device stored in and retrieved from the social health record data bank. The method includes monitoring the first action performed by the first medical device in accordance with the instruction. The method includes instructing the first medical device to pause performing the action for a defined period of time based on an instruction from the processing unit indicative of a second action to be performed by a second medical device.

### BRIEF DESCRIPTION OF THE FIGURES

The features of the disclosed embodiments may become apparent from the following detailed description taken in conjunction with the accompanying drawings showing illustrative embodiments herein, in which:

FIG. 1 illustrates generally, but not by the way of limitation, among other things, an example of an operating environment in which an embodiment may operate;

FIG. 2 illustrates generally, but not by the way of limitation, among other things, a real-time data updating unit such as described in FIG. 1, in accordance with an embodiment;

FIG. 3 is a schematic diagram that illustrates generally, but not by the way of limitation, an exemplary cloud computing architecture, in accordance with an exemplary embodiment;

FIG. 4 illustrates generally, but not by the way of limitation, a plurality of medical devices connected with a social health record data bank (SHRDB), in accordance with an embodiment;

FIG. 5 illustrates, generally but not by the way of limitation, a system for facilitating coordination and control among a plurality of medical devices, in accordance with an embodiment;

FIG. 6 illustrates, generally but not by the way of limitation, an example of a first medical device and a second medical device coordinating in a health care network before switching their operating states, in an embodiment;

FIG. 7 illustrates, generally but not by the way of limitation, an example of the first medical device and the second medical device coordinating in the health care network after switching their operating states, in accordance with an embodiment;

FIG. 8 illustrates an exemplary lookup table depicting interdependence among a plurality of medical devices, in accordance with an embodiment;

FIG. 9 illustrates a method for facilitating coordinated functioning of a plurality of medical devices, in accordance with an embodiment; and

FIG. 10 illustrates generally, but not by the way of limitation, a computer system that may be used in accordance with the embodiments herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

In the following detailed description, reference is made to the accompanying drawings which form a part hereof, and in which is shown by way of illustration specific embodiments in which the embodiments herein may be practiced. These embodiments, which are also referred to herein as “examples,” are described in sufficient detail to enable those skilled in the art to practice the embodiments herein, and it is to be understood that the embodiments may be combined, or that other embodiments may be utilized and that structural, logical, and electrical changes may be made without departing from the scope of the embodiments herein.

A method or a system for dynamically updating real-time data associated with one or more entities of a Social Health Record Data Bank (SHRDB) is provided. The system and method comprises a real-time data updating unit to remotely collect the real-time data associated with the one or more entities over a communication network. The real-time data may include, for example data related to patient, clinician, labs and imaging center, clinical research center, healthcare financing institute, pharmacy, nursing, social services, clinical or medical devices and the like. The real-time data updating unit analyses the collected data to update the one or more records of the SHRDB associated with the one or more entities in real-time. The SHRDB may also run automated monitoring software to alert the one or more entities about the updated records and associated data.

In general, various embodiments provide access to an SHR (social health record) agent, which further allows the one or more users or entities to access and update SHR data. The agent may be accessible directly or indirectly by the entities to manage and update the SHR data. A direct access to the agent can provide quick access to the SHRDB, and an indirect access such as through social service interfaces can also be possible. The SHRDB controls the SHR data based on the predetermined rules and access level associated with the one or more entities such that the one or more entities can access or abstract SHR data associated with various entities from the SHR agent. Accordingly, the agents that lack a configuration to access or update the SHR data directly may instead access SHR data indirectly by calling a healthcare center or any other service that may have an access to the SHRDB or that may authenticate the access based on defined rules and guidelines. The SHR agent can implement one or more electronic security technologies to provide secure SHR data access and updates to the one or more entities, accessing the SHRDB directly or indirectly. The detailed description about these security technologies will be described in later paragraphs of the document.

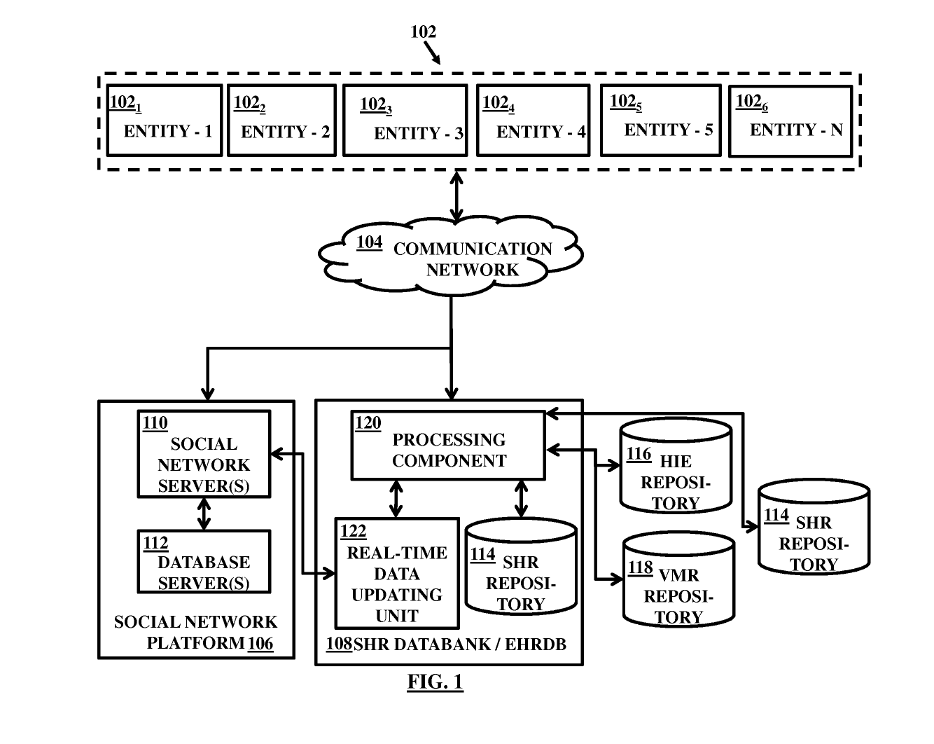

FIG. 1 illustrates generally, but not by the way of limitation, among other things, an exemplary operating environment 100 in which various embodiments may operate. The environment 100 provides a high-level view of one or more entities 102₁₋ₙ (hereafter entities 102 referred, in general) accessing and sharing SHR data in real-time over a communications network 104. The communication network 104 can provide a communicative interconnection of various nodes such as the entities 102, social network platform 106, SHRDB 108, or any other node in the communication network 104. The communication network 104 may include one or more wireless communications network or one or more wire line communications network. The wireless communications network may include for example, but not limited to, a digital cellular network, such as Global System for Mobile Telecommunications (GSM) network, Personal Communication System (PCS) network, or any other wireless communications network. The wire line communications network may include for example, but not limited to, a Public Switched Telephone Network (PSTN), proprietary local and long distance communications network, or any other wire line communications network. In addition, communication network 104 may include for example, digital data networks, such as one or more local area networks (LANS), one or more wide area networks (WANS), or both LANS and WANS to allow interaction among the entities 102. One or more networks may be included in the communication network 104 and may include both public networks such as the Internet, and private networks and may utilize any networking technology and protocol, such as Ethernet, Token Ring, Transmission Control Protocol/Internet Protocol (TCP/IP), or the like to allow interaction among various nodes such as the entities 102, the social network platform 106, the SHRDB 108, or any other node in the network.

The entities 102 described herein may be connected with, for example, any type of electronic data processing system or communication device connected to the communications network 104. Examples of such an electronic data processing system include personal computer systems, such as desktop or laptop computers, workstation computer systems, server computer systems, networks of computer systems, personal digital assistants (PDAs), wireless communications devices, portable devices, or any other electronic data processing system. Likewise, the entities 102 can connect to the communication network 104 through a wired or wireless connection, or a combination thereof, directly or indirectly. An “entity” is understood to mean an individual or a group of individual or an organization, or a platform such as for whom data is managed or who manages data or who facilitate managing of the data in the SHRDB 108. The entities 102 may include for example, but not limited to a patient, clinician or doctor, a lab and research organization, a biller, a hospital, an insurance corporation, an emergency resource such as ambulance, a marketer, an advertiser, an enterprise, a sponsor, an office professional, a social service organization, a social networking platform or any other individual or a group of individuals or an organization or a platform. More generally, each entity 102 can be associated with a unique identifier, usually comprising of a numeric or alphanumeric sequence that is unidentifiable outside the SHRDB 108. In examples, the identifier may also be referred to as a medical record number or master patient index (MPI). The unique identifier can be core of the SHRDB 108 and associated with the entities 102 to link and update all SHR data associated with the entities 102. The detailed description about the SHR data will be described in later paragraphs of the document.

In some embodiments, a social network platform 106, as shown in FIG. 1, may interact with the SHRDB 108 to implement a social cloud among the entities 102. The social network platform 106 can include one or more social network servers 110 and database servers 112 to provide real-time data updates to the SHRDB 108. The social network servers 110 in communication with the database servers 112 may allow the entities 102 to access and update or share SHR data. The social network servers 110 may constantly interact with the SHRDB 108 to provide real-time data updates associated with the entities 102, which are interacting with other entities of the entities 102 or with the SHRDB 108 or with the social network platform 106 in the social cloud. Likewise, the social network servers 110 may also provide real-time updates associated with one or more social services to the SHRDB 108.

The SHRDB 108, described herein, may be centralized or decentralized. The SHRDB 120 may store SHR data related to the entities 102 in an SHR repository 114. The SHRDB 108 may communicate with different servers and repositories such as social network servers 110, database servers 112, Social Heath Care repository (SHR) 114, Health Information Exchange (HIE) repository 116, Virtual Medical Records (VMR) repository 118, or the like to monitor, analyze, and update the SHR data associated with the entities 102. The SHR repository 114 can store the plurality of electronic or social healthcare records including data or information related to the entities 102. The data can be organized in such a way that facilitates local or remote information management in the communication network 104 via a processing component 120. In some embodiments, the processing component 120 may be, but not limited to, a microprocessor, a microcontroller, or equivalent. The processing component 120 may be capable of executing instructions to process data over the communications network 104. In some embodiments, the data corresponding to an individual entity may have been derived from medical testing or treatment or diagnostics reports. In some embodiments, the data may have been derived from other sources such as a research organization trial, insurance services or any other source.

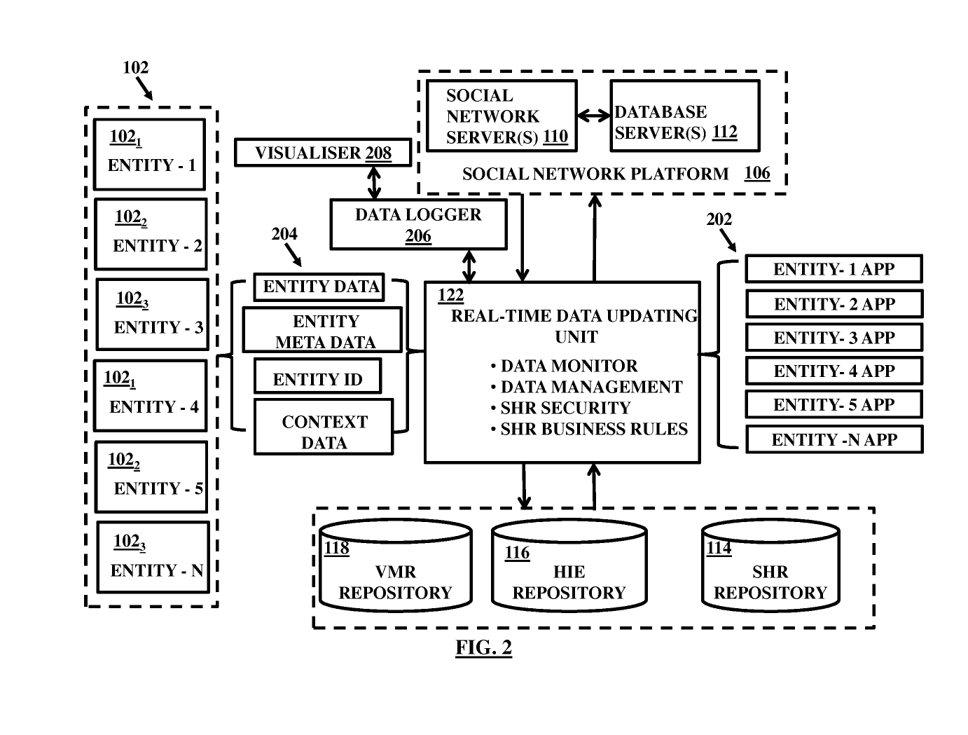

More generally, the SHRDB 108 may also include data related to different electronic or social sources such as doctor's visits, lab tests, hospital stays, clinical trials, patient problems, patients health information, patient habits, patient medical history, patient appointments, patient medical insurance, patient medical bills status, or any other information. The SHRDB 108 may include or couple to other electronic or social data sources such as the HIE repository 116 and the VMR repository 118 to dynamically update information related to or from the other electronic or social sources. The HIE repository 116 may include electronic or social healthcare information related to a region, community, or hospital system. In examples, the HIE repository 116 may provide additional storage, retrieval, and manipulation of health information such that the SHRDB 108 can dynamically manage and update SHR data related to the entities 102. The HIE repository 116 may facilitate mobilization of healthcare information electronically across various repositories connected to the SHRDB 108, across organizations within a region, community or a hospital. The HIE repository 116 may store information or medical records associated with the entities from disparate regions, or communities and allow to electronically move the information or the medical records among disparate health care information systems or repositories of the SHRDB 108. The VMR repository 118 described herein may store electronic or social medical information related to the entities 102 or other sources. The VMR repository 118 may be coupled to the SHRDB 108 to store virtual medical records that may include a simplified, standardized electronic or social health record data designed to support interfacing to the SHRDB 108 or clinical decision support systems. The present system can allow the entities 102 to access and share or update the electronic or social healthcare data in different sources or repositories of the SHRDB 104 such as the SHR repository 114, HIE repository 116, VMR repository 118, or any other sources coupled to the SHRDB 104. The SHRDB 104 may include or be coupled to a real-time data updating unit 122. The real-time data updating unit 122 can be capable of monitoring, collecting, analyzing, and updating the SHR data associated with the entities 102. The detailed description about the real-time data updating unit 122 will be explained in conjunction with FIG. 2.

FIG. 2, with reference to FIG. 1, illustrates generally, but not by the way of limitation, among other things, a real-time data updating unit 122 such as described in FIG. 1, in accordance with various embodiments herein. The real-time data updating unit 122 can be configured to monitor, collect, analyze, and update the SHR data associated with the entities 102. In an example, the real-time data updating unit 122 can be configured to expose a SOAP (Simple Object Access Protocol) based web-service API (Application Program Interface) that allows the entities 102 to post SHR information in real-time directly into the SHRDB 108 such that the real-time data updating unit 122 may analyze the data and transfer a request to the SHRDB 108 to update the corresponding one or more SHR records associated with the entities 102. The above specified protocol is only for exemplary purposes and the present system may use any other protocol capable of managing real-time information from various entities over the communication network 104. As used herein, the term “real-time” may refer to seconds, minutes, or hours, by the way of definition of the particular application, SHRDB 108, or enterprise being controlled and the like. For example, in a certain type of enterprise, the term “real time” may refer to propagating information from the entities within a few minutes or hours. In another enterprise application, the term “real time” may refer to propagating information from the entities substantially immediately, i.e., within a few seconds.

The real-time data updating unit 122 includes various modules such as for example, but not limited to, data monitoring module, data management module, SHR security module, and SHR business rules module. The data monitoring module of the real-time data updating unit 122 monitors the SHR data to discover any updates related to the entities 102. The SHR data described herein may include for example, but not limited to, updates from the entities 102.

The electronic or social health record data or information may include for example, but not limited to, long term care and nursing information, labs and research information, doctor's visits, medication administration records, physician orders, emergency information resource information, hospital stays, clinical trials, patient problems, patients health information, patient habits, patient medical history, patient appointments, patient medical insurance, patient medical bills status, or any other information. The information described herein can be associated with the different entities 102 such as for example patients. This may be referred to as “patient association”. In accordance with some embodiments, several automated and non-automated devices, sensors, and the like devices or equipment can be used in a shared environment like a hospital, or a clinic or any other environment. These devices and sensors, and the like may be used to monitor or record health parameters or information of the entities 102 or patients as an example. For example, these devices can monitor or record information about the blood pressure of patients, and other conditions or status of patients. This monitoring and recording of the patients' information can be performed at several distinct times. For example, a patient may arrive in a hospital in the morning and get his health information recorded or monitored at that time while a second patient arrives in the evening when his health related information is monitored or recorded. In some embodiments, the same devices may be used for monitoring or recording purposes of several patients. The recorded or monitored information is stored in the SHRDB 108 in association with the details of the respective patients. This may allow the SHRDB 108 to know which data elements or detail corresponds to which patient. The patient or entities association may allow the SHRDB 108 to function in a proper and accurate manner. The SHRDB 108 can easily trace records for specific patients through a “patient association” technique.

The real-time data updating unit 122 is configured to include and use information about one or more entity applications 202₁₋ₙ (hereafter generally referred as entity applications 202) to update the real-time data associated with the entities 102 via the data management module. The entity applications 202 described herein may include for example, but not limited to, patient application, clinical application, lab application, admin application, health care application, financial organization application, insurance application, research organization application, or any other entity application. The real-time data updating unit 122 can be configured to use the data management module to update the real-time data identified by the data monitoring module. The real-time data updating unit 122 may update the data and SHRs of the entities 102 based on the information propagated from the data monitoring module of the real-time data updating unit 122. The real-time data updating unit 122 may use identification information 204 associated with the entities 102 to update the corresponding entity applications 102 and electronic or social healthcare records. The identification information 204 described herein, may include for example, but not limited to, entity specific information such as entity name, age, gender, phone number, contact details and the like, entity meta data such as to quickly and precisely search for desired SHR data and related entities 102, entity identifier (ID) such as an entity unique identifier, entity specific contextual data such as research notes pertaining to the entity, or any other identification information. The real-time data updating unit 122 may use identification information 204 associated with the entities 102 to identify one or more corresponding electronic or social health records to be updated. This may include, for example, a mobile phone number, IP address of a computer and any other reference associated with the entities 102.

The real-time data updating unit 122 may send a request to the SHRDB 108 to update the corresponding data or SHRs, in accordance with the identified real-time data associated with the entities 102. Likewise, in some examples, the SHRDB 108 may allow the real-time data updating unit 122 to interact with other servers and repositories such as the social network servers 110, the database servers 112, the SHR repository 114, the HIE repository 116, the VMR repository 118, and the like to monitor and update SHR data propagated to or from the social cloud. The real-time data updating unit 122 identifies the real-time data and send a request to the SHRDB 108 for dynamically updating the associated SHRs and other data sources such as the HIE repository 116, and the VMR repository 118. The SHRDB 108 may run automated monitoring software to alert the entities 102 about the updated records and associated data.

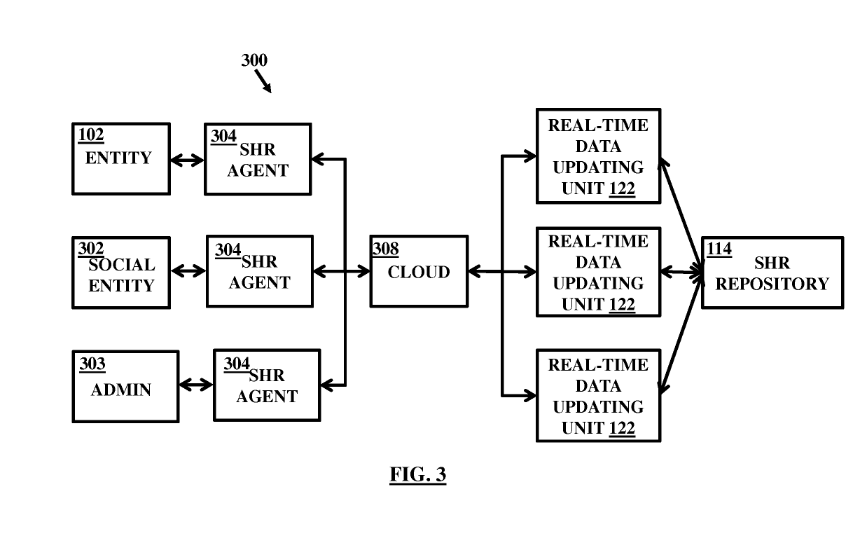

The SHR security and business rules module may implement one or more security technologies and pre-determined business rules to provide secure access and updates to or from the SHRDB 108, the SHR repository 114, the HIE repository 116, and the VMR repository 118 such that the entities 102 can access and update the SHR data, based on the entities role and access levels, over the communication network 104, directly or indirectly via the SHR agent such as described in FIG. 3. In some embodiments, the real-time data updating unit 122 is coupled to a data logger 206, as shown in FIG. 2. The data logger 206 is configured to retrieve data from various devices integrated or coupled or included to/within the SHRDB 108, and log the data in a single repository. The single repository can be the SHRDB 108. The data logger 206 is coupled to a visualizer 208 that is configured to retrieve the data from the data logger 106 or SHRDB 108 and visualize it such that a health care-specific task can be operated on it. In some embodiments, the visualizer 208 can generate visual reports that may or may not be tabulator in nature. The visualizer 208 provides an output that is simplified for a physician to make sense thereof.

FIG. 3, with reference to FIGS. 1 and 2, is a schematic diagram that illustrates generally, but not by the way of limitation, an exemplary cloud computing architecture 300, in accordance with the various exemplary embodiments herein. An entity 102 can access the SHR data via an SHR agent 304. The SHR agent 304 described herein may be a software component running on the electronic data processing system of the entities 102. The SHR agent 304 can be a web-based agent that may allow access to the SHR data associated with the entities 102. The web-based agent may be accessed directly or indirectly. The direct access to the web-based agent can be done by the entities 102 via a Uniform Resource Location (URL) address. The web-based agent can also be accessed indirectly by various social entities 302 via a social service interface or any other third party interface. The social entity 302 described herein can be, but not limited to, a general user of the social service such as Facebook™, Orkut™, LinkedIn™ Twitter™, or any other social service. In an example, the SHR agent 304 may be a standalone application configured to be installed on the electronic data processing systems of the entities 102. The SHR agent 304 can be configured to provide access to the various application modules of the SHRDB 108, based on the entities role and access level.

In an example, the entity 102 may update the SHR data via the SHR agent 304 in real-time over the cloud 308 facilitated by the communication network 104. The real-time data updating unit 122 may monitor the real-time information updates to or from the entities 102 to identify corresponding entities records to be updated. The real-time data updating unit 122 may transfer a request to update the one or more entities records and applications in the SHR repository 114, in accordance with the propagated real-time information. In another example, where the SHR agent 304 may be accessed via a social entity 302, the real-time data updating unit 122 monitors the real-time information to or from the social entities 304 to identify corresponding entities records to be updated. Likewise, the real-time data updating unit 122 may also monitor the updates to or from the social service providing access to the social entities 302 to update SHR data over the social cloud. The real-time data updating unit 122 may also monitor the real time updates to or from the administrators 303 or the electronic healthcare providers and updates corresponding electronic or social healthcare records associated with the entities 102.

A method for dynamically updating electronic or social healthcare data associated with the entities 102 in the SHRDB 108, in accordance with various embodiments, can also be employed. The real-time data updating unit 122 of the SHRDB 108 monitors real-time information updates from the entities 102. The real-time data updating unit 122 identifies the one or more electronic or social healthcare records to be updated, in accordance with the propagated real-time information. The real-time data updating unit 122 transfers a request to update the corresponding electronic or social healthcare records associated with the entities 102.

The methods described herein may be deployed in part or in whole through a machine that executes software programs on a server, client, or other such computer and/or networking hardware on a processor. The processor may be part of a server, client, network infrastructure, mobile computing platform, stationary computing platform, or other computing platform. The processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon.

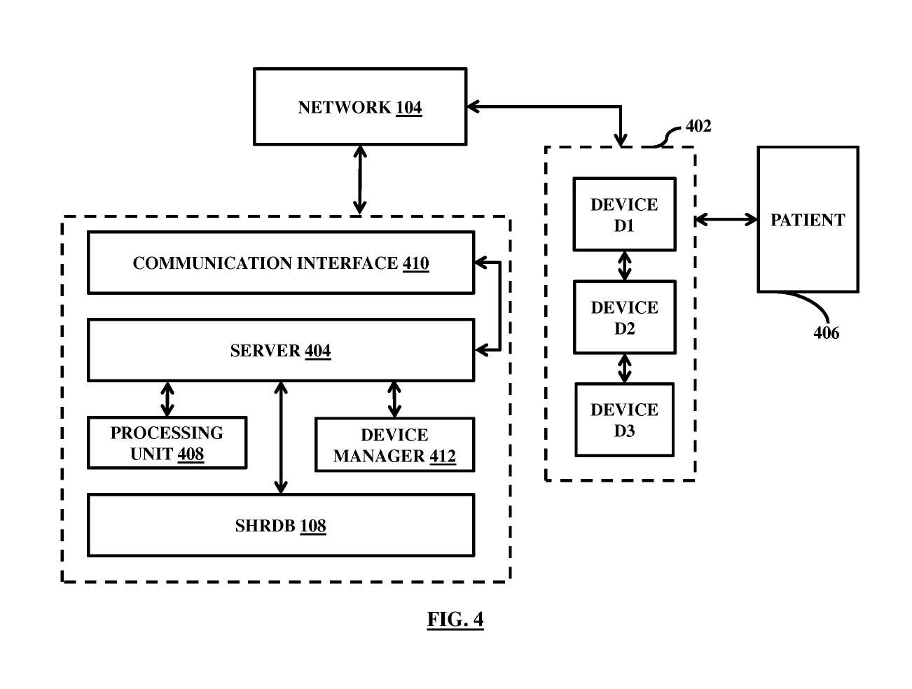

In accordance with some embodiments, the EHRDB 108 can control other devices (D1, D2, D3) operating such as in a medical environment as shown in FIG. 4 and discussed later.

In some embodiments, the EHRDB 108 and the devices can be integrated to provide the “device integration” functionality as discussed later. In some embodiments, the several devices such as D1, D2, and D3 can be integrated among themselves also while simultaneously coupled or integrated to the EHRDB 108. In accordance with the device integration functionality, a device may be associated with a patient and the monitored or recorded data can be sent to the EHRDB 108. The data sent may, for example, be sent from a specific device such as D1 or D2 and the like.

In accordance with some embodiments, a device such as D1 may be provided or used for a patient. In an example, if the patient is associated with the particular device D1, and when the EHRDB 108 is used in proximity of the device D1, EHRDB 108 automatically begins functioning in the context of the particular patient. In an embodiment, software can also be used to automatically connect to the patient simply based on the device D1 that the patient is connected or associated with.

In some embodiments, the EHRDB 108 may provide a functionality of device (D1, D2, and D3) coordination. In accordance with the functionality of “device coordination”, based on certain kinds of information received from a device such as blood pressure from a blood pressure device, a gateway can send a command to the EHRDB 108 or to other devices to do some specific task. For example, a ventilator may be required to be turned off if an X-ray is taken during a surgical procedure. So, a message is required to be sent to the gateway and other related devices to turn the ventilator off while the X-ray is taken and once the X-ray has been taken, a command to switch on the ventilator is required to be given. The device coordination functionality allows the EHRDB 108 and the devices (D1, D2, and D3) to perform these tasks in a proper and coordinated way.

In still some other embodiments, the EHRDB 108 may provide a functionality of device (D1, D2, and D3) control. In an embodiment, if a device such as D1 is employed at a particular location within the medical environment, the EHRDB 108 may show a button to indicate the device D1, and its association with the patient. For example, if the device D1 is a BP cuff tied to a patient, the EHRDB 108 may indicate the location of the cuff and the association of the cuff with the patient by providing a button on a screen or user interface of the EHRDB 108 that is configured to be pressed. As soon as the button is pressed on the user interface, the EHRDB 108 sends a command to the cuff invoking it to measure the BP. A physiological sensor coupled to the patient or the cuff can send the recorded or monitored data to the EHRDB 108. Thus, the “device control” functionality may provide the EHRDB 108 to control the devices D1, D2, and D3 coupled to the EHRDB 108.

In some embodiments, software may be provided that can automatically send a command to press the button on the user interface. By pressing the button, the device can automatically perform a desired task and send the monitored or recorded data to the EHRDB 108.

In some embodiments, the device (D1, D2, D3), can be a phone, a tablet, a computer or any other device that contains sensors, an accelerometer, GPS enabled devices, and the like. The various functionalities of the medical devices are further discussed below in detail.

It must be appreciated that various embodiments as discussed above in conjunction with FIGS. 1-3 may be combined with various embodiments implementing device integration, device coordination, and device control functionalities as disclosed below in conjunction with FIGS. 4-9.

FIG. 4, with reference to FIGS. 1 through 3, illustrates a plurality of medical devices 402 (D1, D2, D3) connected with the EHRDB 108 through the network 104. The EHRDB 108 may also be referred to as SHRDB (Social Health Record Data Bank) 108 interchangeably without any limitation throughout this document. The EHRDB 108 stores data or medical records of a plurality of patients electronically. The medical records may include one or more of demographic information, medical history, treatment plans, ongoing treatments, information related to allergies, and lab reports of the plurality of patients, and the like. In accordance with some embodiments, the medical records of the plurality of patients stored in the SHRDB 108 can be obtained socially through social aware networks linked to the respective plurality of patients. The medical records may also be referred to as social health records herein as they may be obtained from various socially aware networks.

The SHRDB 108 may be coupled to a server 404. The plurality of medical devices 402 may be coupled to the server 404 either locally or remotely so as to control functioning and coordination of the plurality of medical devices 402 based on information stored in the SHRDB 108. The stored information may be associated with the plurality of patients. For example, the SHRDB 108 may collect data about the patients and the associated devices 402 from various socially aware networks for example social networking websites and accordingly based on the data stored in the SHRDB 108 can control and coordinate functioning of the devices 402. The stored data may be updated by the real time updating unit 122 in real time. In an embodiment, the plurality of medical devices 402 may be integrated among themselves and also with the server 404. In an embodiment, the functioning of the plurality of medical devices 402 may be controlled or coordinated with respect to one another by the server 404. In an embodiment, the functioning of the medical devices 402 may be coordinated or controlled with respect to one another in a defined sequence. For example, the device D2 may operate after device D1 stops functioning for a defined period of time. The device D3 may operate only after D2 stops functioning for a defined period of time. Even more than one device can operate simultaneously based on the defined sequence by the server 404. The defined sequence can be determined by the server 404 based on for example information pertinent to a patient 406 associated with the plurality of medical devices 402, information about the devices 402, functioning of the devices 402, medical condition of the patient 406, and level of requirement of the devices 402 for the patient 406 under the given medical condition of the patient 406.

The server 404 may send instruction to the plurality of medical devices 402 associated with the patient 406 for proper coordination and control of the devices 402. The server 404 may include or be coupled to a processing unit 408. The server 404 may further include a communication interface 410 to communicate with the medical devices 402.

In an embodiment, the SHRDB 108 and the server 404 may be deployed in a hospital facility to control and coordinate with medical devices associated with patients in the entire hospital hospitality. In such embodiments, the SHRDB 108 may be connected to hospital information systems to socially collect data from various information sources within the hospital. The SHRDB 108 may further receive medical records associated with the patients from outside the hospital environment through for example various socially aware networks. The socially aware networks may include various social networking websites that the patients may be associated or registered with. The SHRDB 108 may be accessed by authorized users of the hospital facility through a web based platform. In an embodiment, the SHRDB 108 and the server 404 may be deployed to communicate with several hospital facilities and organizations such that the SHRDB 108 may store information pertinent to patients and medical devices of the entire hospital facilities and the organizations.

In an exemplary embodiment, the server 404 may connect with a plurality of medical devices in a hospital facility or any other medical environment even including multiple hospital facilities and organizations and detect and locate a medical device in the health care environment, initialize with the medical device and after initialization may remotely network with the medical device such that the SHRDB 108 may be able to transmit and receive information from the medical device. For example, this may be done by connecting the SHRDB 108 and the server 404 to Internet or other communication network or communication system.

In an exemplary embodiment, the server 404 can remotely monitor and diagnose the plurality of medical devices 402 associated with the patient 406. The server 404 can allow remote monitoring and diagnostics of a remotely located device from among the plurality of medical devices 406 once the medical device has been located and analyzed by the server 404 and relevant details about the device and the patient 406 associated with device are obtained and stored in the SHRDB 108. In an example, the server 404 may connect to any of the plurality of medical devices 402 located within any health care facility and/or outside health care facility.

In an embodiment, the server 404 may be coupled to a device manager 412. The device manager 412 may be configured to manage device information corresponding to each of the plurality of medical devices and associate the information with respective patients. For example, the device manager 412 may be responsible for managing the information of the devices D1, D2, and D3 and associate this information with the patient 406. In this manner, the processing unit 408 along with the device manager 412 can perform tasks such as monitoring and coordinating and controlling of the medical devices 402 in a health care facility. In some embodiments, the device manager 412 may be configured to detect newly connected medical devices or any updates in the existing medical devices associated with an already registered patient with the SHRDB 408. For example, the device manager 412 can detect and locate a medical device D1 and identify the medical device D1 and authenticate it and provide an administrator or privileged access to the information relevant for the device D1 in the SHRDB 108. The relevant information may for example be information pertinent to other medical devices such as D2 and D3 that coordinate with the newly detected medical device such as D1.

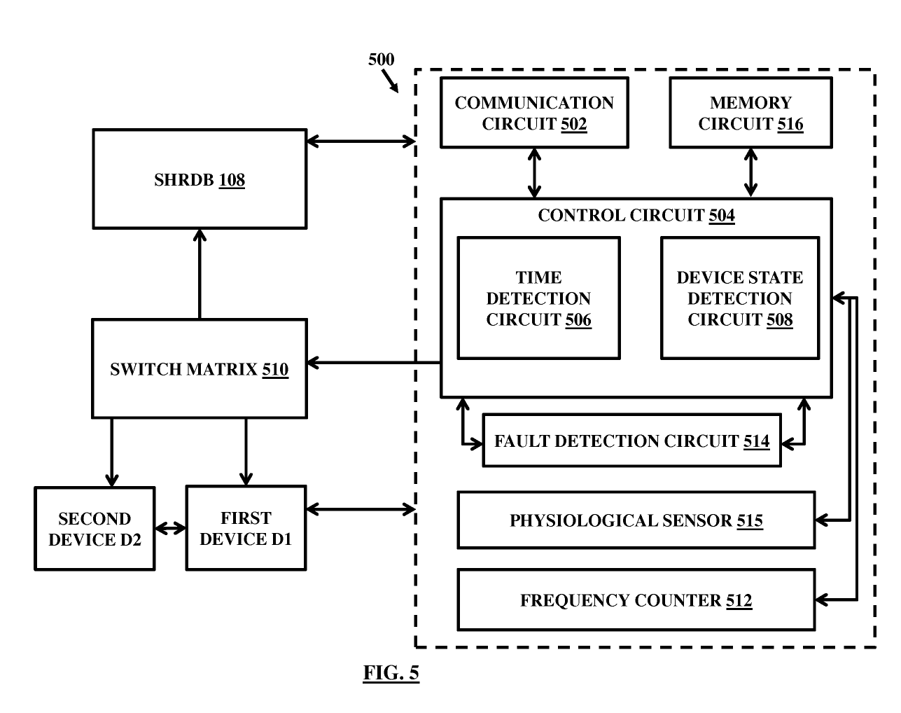

In accordance with various embodiments, a system is provided that is configured to be communicatively coupled with the SHRDB 108, server 404, and the plurality of medical devices 402. The system may facilitate communication between the server 404 and the plurality of medical devices 402. The system may be responsible for managing the medical devices 402 associated with the patient 406 and facilitate coordinated functioning and integration and control of the plurality of medical devices 406 by providing a channel therethrough. The system is described later in conjunction with FIG. 5 below.

In accordance with some embodiments, the devices 402 can include, for example, a blood pressure (BP) cuff, X-ray machine, a ventilator, Electrocardiogram (ECG) device, radiotherapy device, dosing controller, medical resonance therapy related device, and the like devices.

FIG. 5, with reference to FIGS. 1 through 4, illustrates a system 500 for facilitating coordinated functioning of the plurality of medical devices 402 over the network 104. The system 500 may be coupled to the server 404 and the plurality of medical devices 402. The system 500 may facilitate communication between the server 404 and the plurality of medical devices 402. The system 500 may manage the medical devices 402 associated with the patient 406 and facilitate coordinated functioning and integration and control of the plurality of medical devices 402 by providing a channel therethrough.

The system 500 may include a communication circuit 502, and a control circuit 504. The communication circuit 502 may be configured to receive an input from the server 404 or the processing unit 408 coupled to the server 404. The input may be indicative of an action or a task to be performed by a first medical device such as D1 from among the plurality of networked medical devices 402. In an embodiment, each of the plurality of medical devices 402 may perform a unique task and each of the tasks performed by the plurality of medical devices 402 may be coordinated and dependent with respect to one another. The interdependence may be known to the server 404 and information about the interdependence of the tasks may be stored in the SHRDB 108 in association with patient information corresponding to the medical devices 402. For example, a particular task performed by the first medical device D1 may not be desired along with the second task performed by the second medical device D. In such cases, the server 404 sends the instruction to the system 500 which is received by the communication circuit 502 as the input. The received input by the communication circuit 502 provides a guidance that allows the medical devices 402 to function in a defined and desired manner and in accordance with the interdependence already known and stored in the SHRDB 108. The interdependence may be decided by doctors or experts or persons treating the patient, or based on medical facts. The patient or the doctors or the persons treating the patient may convey the interdependence to the SHRDB 108 through various channels such as through socially aware networks. In an embodiment, the information about tasks interdependence may be automatically updated with the SHRDB 108. In an embodiment, the SHRDB 108 may automatically determine the information about the tasks interdependence as and when any medical device is registered with the SHRDB 108 in association with a patient. The SHRDB 108 may for example receive device related information and accordingly map the device information with other coordinating medical devices to define the interdependence. Once, the task interdependence is determined and known to the server 404, the functioning of the medical devices 402 may be coordinated and controlled accordingly based on the interdependence. The terms ‘task’ and ‘action’ are used herein throughout the draft interchangeably without any limitations.

The communication circuit 502 may be configured to send an instruction to the first medical device D1 to initiate the first task by the first medical device D1. The instruction may also include information about the details of the first medical device D1. In an embodiment, when the first medical device D1 functions for more than one patient, the instruction may also include information of the patient 406 associated with the first medical device D1. The information about the first medical device D1 and the patient 406 corresponding to the first medical device D1 may be stored in and retrieved from the SHRDB 108.

The control circuit 502 may be configured to monitor the task performed by the first medical device D1 and receive an update periodically. In an event of a requirement of a second task performed by the second medical device D2, the system 500 may enquire the server 404 about interdependence of the first task and the second task and about the interdependence of the first medical device D1 and the second medical device D2. In an embodiment, the server 404 may retrieve the information about interdependence from a lookup table stored in the SHRDB 108. The processing unit 408 may process information about the interdependence. In case, there is a conflict between the first task by the first medical device D1 and the second task by the second medical device D2, the server 404 may send the information about conflict to the system 500 accordingly. In another embodiment, the system 500 may not enquire about the interdependence rather instructions for performance or non-performance of the tasks are sent by the server 404 to the system 500.

The control circuit 504 may be configured to instruct the first medical device D1 to pause performing the first task for a defined period of time based on an instruction from the server 404 after determining tasks' interdependence information. The instruction may also be indicative of the second task to be performed by the second medical device D2. The predefined period of time may depend on various factors such as patient information, nature and type of the first and second medical devices D1 and D2, functioning and behavior of the first and second medical device D1 and D2, medical condition of the patient 406, degree of requirement and necessity of the first and second tasks for the patient 406 in light of the current medical condition of the patient 406. For example, if the second task is extremely necessary and vital for the current medical condition of the patient 406, the instruction may indicate to perform the second task for a time necessary to control criticality of the medical condition of the patient 406.

The control circuit 504 may include a time detection circuit 506 configured to monitor the defined period of time during which the first medical device D1 pauses performing the first task and the second medical device D2 performs the second task. The time detection circuit 506 may be configured to generate a signal upon completion of the defined period of time. The signal may be indicative of resuming the first task by the first medical device D1 and stopping of the second task by the second medical device D2. The time detection circuit 506 may be configured to initialize at a zero time value and monitor the passage of time until the monitored time equals the defined period of time after which the signal is generated indicative of the resuming of the first task by the first device D1 and stopping of the second task by the second device D2.

The control circuit 504 may include a device state detection circuit 508 coupled to the time detection circuit 506 and configured to identify an operating state of the plurality of medical devices 402. The operating state may be defined by performance or non performance of the medical devices 402. For example, the performance of the first medical device D1 may define a first operating state and non-performance of the first medical device D2 may define a second operating state of the first medical device D1. Similarly, the performance of the second medical device D2 may define a first operating state and non-performance of the second medical device D2 may define a second operating state of the second medical device D2. Based on interdependence between the first and the second tasks, the operating states of the first medical device D1 may depend on the operating states of the second medical device D2. For example, if the interdependence suggests a conflicting situation between the first and the second tasks, the first operating state of the first medical device D1 may not occur during occurrence of the first operating state of the second medical state D2. Therefore, either of the first and second medical devices D1 and D2 has to be in a non-performing operating state during the defined period of time.

The system 500 may include a switch matrix 510 that is configured to cause a switching action of the operating states. For example, the switching matrix 510 may be configured to cause switching of the first and the second medical devices D1, D2 from a performing operating state to a non-performing and from a non-performing state to a performing state based on an instruction from the control circuit 504. In an embodiment, the switch matrix 510 may cause switching of the operating states of one or more of the first medical device D1 and the second medical device D2 upon receipt of the instruction from the control circuit 504. In an embodiment, the switch matrix 510 may cause switching of the operating states of one or more of the first medical device D1 and the second medical device D2 upon receipt of the signal generated by the time detection circuit 506 that is indicative of completion of the defined time and of resuming the first task and stopping the second task. The resuming of the first task requires a switching of the non-performing state of the first medical device D1 to a performing state of the first medical device D1. The stopping of the second task by the second medical device D2 requires switching of the performing state to the non-performing state of the second medical device D2 by the switch matrix 510.

The control circuit 504 may include or be coupled to a frequency counter 512 configured to determine frequency of switching of operating states of the medical devices 402. For example, the frequency counter 512 may be configured to determine frequency of switching of the first medical device D1 from a performance state to a non-performance state of the first action and vice versa. Similarly, the frequency counter 512 can be configured to determine frequency of switching of operating states of other medical devices such as D2. In an embodiment, the frequency counter 512 may generate an output indicative of a current frequency of switching of an operating state of a medical device such as D1 and D2. The frequency counter 512 may further be configured to map the current frequency of switching with a threshold frequency established by the SHRDB 108 based on a set of physiological parameters in association with the information associated with the patient 406. In an embodiment, the physiological parameters may include vital signs of the patient 406. The relevant information of the patient 406 may include for example without limitations degree of necessity of switching of the operating states of one device with respect to other device for the patient 406, age, gender, medical condition and other such patient specific information. For example, an aged person in a critical condition may not tolerate frequent putting off of a ventilator machine and therefore a person responsible for patient care may not allow switching of the ventilator to an off state very frequently beyond a threshold limit. In an embodiment, the threshold limit may be defined for a unit period of time. For example, switching of a patient's ventilator from an on to off state may in certain conditions be not allowed beyond three times a day with each not more than 15 minutes. The frequency counter 512 may further be configured to reject the switching of the operating state of the first medical device D1 or the second medical device D2 if the current frequency of switching equals the threshold frequency for the respective medical devices D1, D2. For example, upon receipt of the instruction by the communication circuit 502 to switch the first medical device D1 from the performing state to the non-performing state for the defined period of time, the request for switching may be rejected by the frequency counter 512 if the current frequency already reached the threshold frequency.

In accordance with various embodiments, there should be a consistency in output generated by the time detection circuit 506 and the device state detection circuit 508. For example, upon completion of the defined time and its detection by the time detection circuit 506, there should be a change in an operating state of the first medical device D1 from a non-performance to a performance state. Therefore, both the time detection circuit 506 and the device state detection circuit 508 should lead to a state of shift which should be consistent. In an event of an inconsistency, a fault may occur. The system 500 further includes a fault detection circuit 514 configured to generate a signal indicative of a fault if the device state detection circuit 508 does not detect a change in the operating states of either of the first and second medical devices D1, D2 upon receipt of the instruction from the controller circuit 504 to initiate or pause the first action and upon generation of the signal by the time detection circuit 506 to stop the second action and resume the first action on completion of the defined time. The signal generated by the fault detection circuit 514 may be transmitted to the SHRDB 108 through the communication circuit 502 to determine optimal states of the first medical device D1 and the second medical device D2 upon detection of the failure. The optimal states can be other than what is initially instructed by the server 404 to the system 500 for example to switch the first medical device D1 to non-performing state and to switch the second device D2 to performing state for the defined time. The server 404 may be configured to determine the optimal states of the first and second medical devices D1, D2 based on the information of the patient 406 associated with the first medical device D1 and the second medical device D2. For example, the server 404 may determine an alternative treatment technique to fulfill the requirement that is not achieved because of the detection of the fault. In an embodiment, the server 404 can send an instruction to the system 500 to guide a patient caretaker to manually attend the patient 406. There can be several reasons for the fault. One reason may be malfunctioning of the first and/or the second medical device D1, D2. In such cases, the defaulted medical device may be replaced. Another reason may be a rejection by a medical device to follow the instruction sent by the control circuit 504 or the switch matrix 510 to switch the operating states. The rejection may occur because at least one medical device may be authorized to reject the instruction in some embodiments. For example, the first medical device D1 may be authorized to reject to pause performing the first task based on the instruction received from the control circuit 504. The rejection by the first medical device D1 influences the second task to be performed by the second medical device D2. In such cases, the first medical device D1 may coordinate with other medical devices but may not be completely controlled by the system 500 to function in a defined manner. This is because the first medical device D1 is provided a privilege to reject the request for switching the state. In other embodiment, however, the first medical device D1 may not be authorized to reject the request for switching and may be completely controlled by the system 500. In an event of rejection, the server 404 may be informed about the rejection to determine an alternative action for the second medical device D2 or an alternative medical device to replace the need of the second medical device D2 by a third device to perform a third task (that may be similar or alternative to the second task) in a manner that the first medical device D1 has no objection with the third task. For example, the first medical device D1 may not reject because the third action may be defined in a manner that it may not need pausing of the first task, and the first and the third tasks may be performed simultaneously because of a specific interdependence between them. The server 404 may in some embodiments determine the alternative tasks or medical devices with the use of the interdependence information saved in the SHRDB 108 or in a memory of the system 500.

In some embodiments, the system 500 may include a physiological sensor 515 configured to be coupled to the patient 406 to identify a set of physiological characteristics of the patient 406 during performance and non-performance of the first and the second medical devices D1, D2. The physiological characteristics may be one or more of blood pressure, heart beat, respiratory rate, body temperature, and glucose level, and other such characteristics without limitations. In an example, the switching of the operating state for example from performance to non performance or vice versa of the first and second medical devices D1, D2 based on the instruction received from the server 404 may be rejected upon indication of a change in the physiological characteristics beyond respective threshold values corresponding to each of the physiological characteristics. The physiological sensor 515 may determine indicative values of the one or more physiological characteristics of the patient 406 before switching and after switching, and upon a change in the characteristics beyond the threshold limit, the request for switching from the server 404 may be rejected. In an embodiment, the physiological sensor 515 may forecast possible changes that may occur in the physiological characteristics after switching based on the patient specific information, information about the characteristics and other medical information stored in the system 500. Based on the determined values of the physiological characteristics before switching and possible forecast values after switching, the request for switching may be rejected if the change in the values of the physiological characteristics is beyond the threshold limit. In an embodiment, the physiological characteristics may include without limitations blood pressure, heart beat, respiratory rate, body temperature, and glucose level. The physiological sensor 515 may be coupled to the patient 406 for monitoring the various physiological characteristics. In an embodiment, the physiological sensor 515 may be implanted within a patient body. In an embodiment, the physiological sensor 515 may be implanted as a standalone component. In another embodiment, the physiological sensor 515 may be implanted as a component of another implantable device already implanted in the body such as a pacemaker or a defibrillator, or any other implantable device.

In an embodiment, the threshold values of the physiological characteristics may be determined and updated regularly by the processing unit 408 of the server 404 and stored in the SHRDB 108 in real time. The updating of the threshold values can depend on an update in patient specific information such as age, disease, gender, and height-weight index of the patient 406 and the like information and are communicated to the system 500 for storage locally within the system 500 in real time. The system 500 may include information about the threshold values of the physiological characteristics in a memory circuit 516. The memory circuit 516 may further store the information received from the processing unit 408 coupled to the server 404 and patient-specific information obtained from the SHRDB 108.

In an embodiment, the SHRDB 108 may be configured to store medical records of a plurality of patients including the patient 406. The medical records may be associated with the plurality of patients and may be obtained socially through social aware networks linked to the respective plurality of patients. For example, the patient 406 may register him with a social networking website and upload his medical information and the server 404 may retrieve information from the website and store in the SHRDB 108. The SHRDB 108 may function as a two way communication database which can access federated records from socially aware networks of the patients for collecting and collating the medical records and also serve as a repository to be accessed by the patients from anywhere to view and access their records at a single location. In various embodiments, the medical records may include one or more of demographic information, medical history, treatment plans, ongoing treatments, information related to allergies, and lab reports of the plurality of patients.

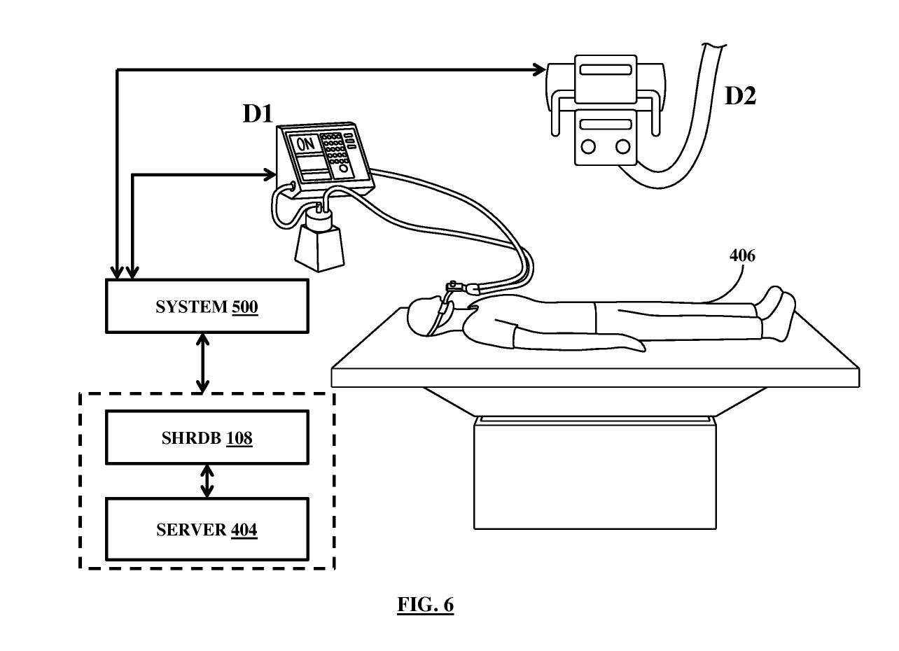

FIG. 6, with reference to FIGS. 1 through 5, illustrates an exemplary embodiment of the medical devices D1 and D2 coordinating through and controlled by the system 500. As shown, the first medical device D1 is a ventilator and the second medical device D2 is an X-ray machine. The ventilator D1 is shown in the first operating state which is defined by performing the first task that is providing artificial life support to the patient 406. The second medical device D2 is shown in the second operating state which is defined by non-performing the second task by the second medical device D2. The first task may be dependent on the second task. As shown, the interdependence between the first task and the second task is such that when the first medical device D1 operates to provide artificial ventilation, the second medical device D2 stays off. Therefore, the X-ray D2 machine is shown as in an off state or in the second state of non-performing of the task of radiating to capture an X-ray. This interdependence may be established based on well known medical reasons that an X-ray should not be taken while a patient is on a ventilator machine. Similarly, in some other examples, the interdependence can be established based on various other factors also such as discussed above.

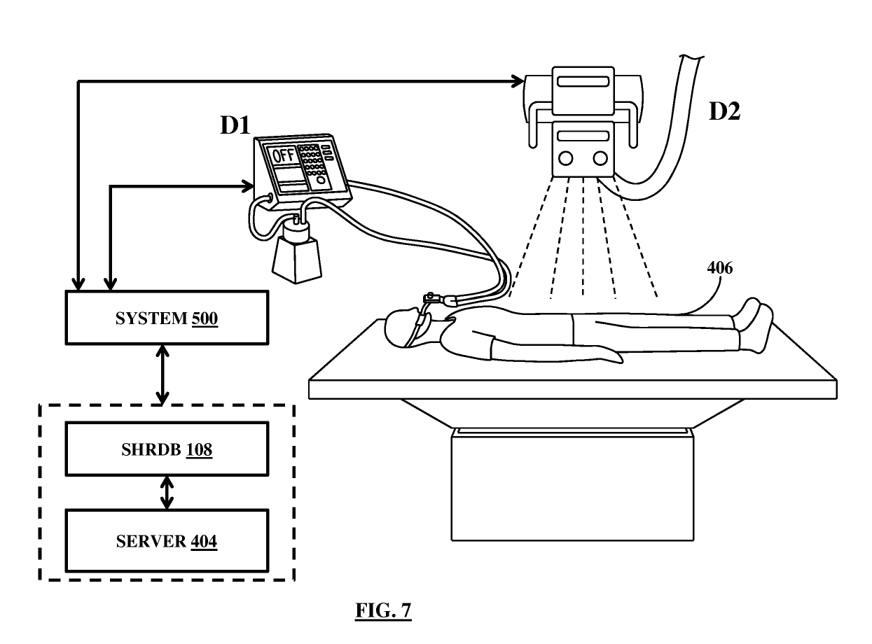

FIG. 7, with reference to FIGS. 1 through 6, shows the exemplary embodiment of FIG. 6 after switching of the operating state of the first medical device D1 based on the instruction received from the switch matrix 510 or the control circuit 504. The interdependence between the ventilator D1 and the X-ray machine D2 is of the type that the X-ray machine D2 will operate only when the ventilator D1 is off. Therefore, the instruction of switching includes sending a request to the ventilator D1 (first medical device) to pause delivering the artificial ventilation and sending a request to the X-ray machine D2 (second ventilator) to activate its task that is delivering radiation for capturing the X-ray. The FIG. 7 therefore shows the ventilator D1 in an off state for the defined time and the X-ray machine D2 in an on state for the defined time. The defined time may be based on various factors as discussed above. One of them may be the level of necessity of the ventilation by the patient 406. For example, if the patient 406 is under critical condition, ventilation may be paused for a very short period of time only. If the patient 406 is in an extremely critical condition, then the task of taking the X-ray may altogether be postponed or rejected. The server 404 may be updated accordingly in case of the postponement or rejection. The SHRDB 108 may maintain a log of such rejections or postponements for the patient 406 so as to accordingly refine the interdependence of the various tasks performed by various medical devices associated with the patient 406 for future use. For example, in the future for a defined time such as three days, the system 500 may not instruct the X-ray machine D2 to operate in view of the refined interdependence established and stored in the SHRDB 108 when the ventilator D1 is on.

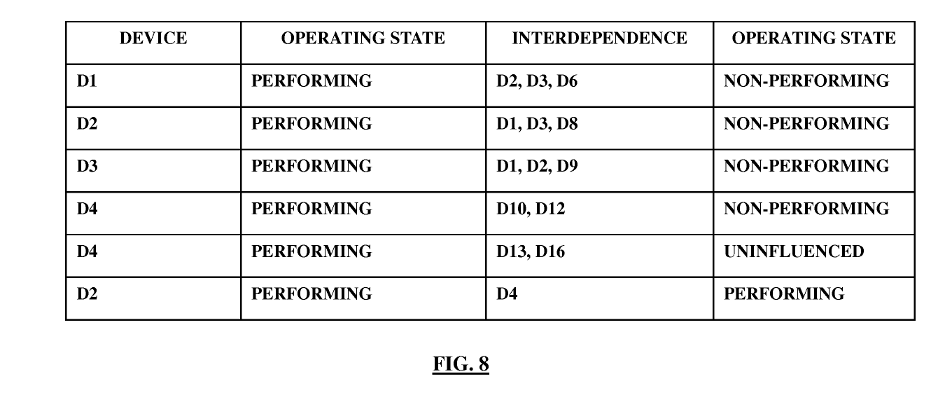

FIG. 8, with reference to FIGS. 1 through 7, illustrates exemplary listing of interdependence in the look up sheet maintained by the SHRDB 108. It must be noted that the interdependences are dynamic in nature and are updated after periodic intervals or in real-time. The real time updating can be done with the use of the real time data updating unit discussed in FIG. 2. As shown, in examples, when D1 performs its task, D2, D3, and D6 cannot perform or operate in their performing state. When D2 operates in the performing state, D1, D3, and D8 cannot perform. When D3 performs D1, D2, and D9 cannot operate to perform. When D4 performs, D10, and D12 cannot perform. When D4 performs, D13, and D16 remain uninfluenced that is whether D13 or D16 or both perform or not, they do not affect operating state of the D4. When D2 performs, D4 has to perform.

In some embodiments, priority of performance by different medical devices may be established. For example, if the device D1 cannot perform with the devices D2, D3, and D6, then which devices(s) should perform with a higher priority. This may be determined dynamically based on several factors for example as discussed above including without limitation patient current medical condition, affect of performance or non-performance of a device on the patient medical condition, nature and functioning of the devices and their relationship with the medical condition of the patient, and the like. For example, if it is determined that D2 is more needed by the patient and gets a higher priority over D1, then D1 will be put in an off state that is non-performing state and D2 starts performing. A device that may possess a higher priority with respect to a particular device may possess a lower priority with respect to another device.

It must be appreciated that though the above discussion includes various embodiments that involve the use of two medical devices D1 and D2, it must be appreciated that more than two medical devices may also be employed equally without limiting the invention. For example, the system 500 may facilitate coordinated functioning of the plurality of devices (two or more than two) coupled to the system 500. The SHRDB 108 may store the medical records of the patient 406 and information related to a sequence of operations to be performed by the plurality of the medical devices based on interdependence among the plurality of the medical devices, identification information of the devices, and patient specific information. The server 404 may use the stored information to guide the system 500 to instruct the plurality of devices accordingly in the defined sequence. It must be appreciated that the though the above discussion and the figures include one patient associated with medical devices, several patients each associated with one or more devices may also be coordinated and controlled accordingly without any limitation.

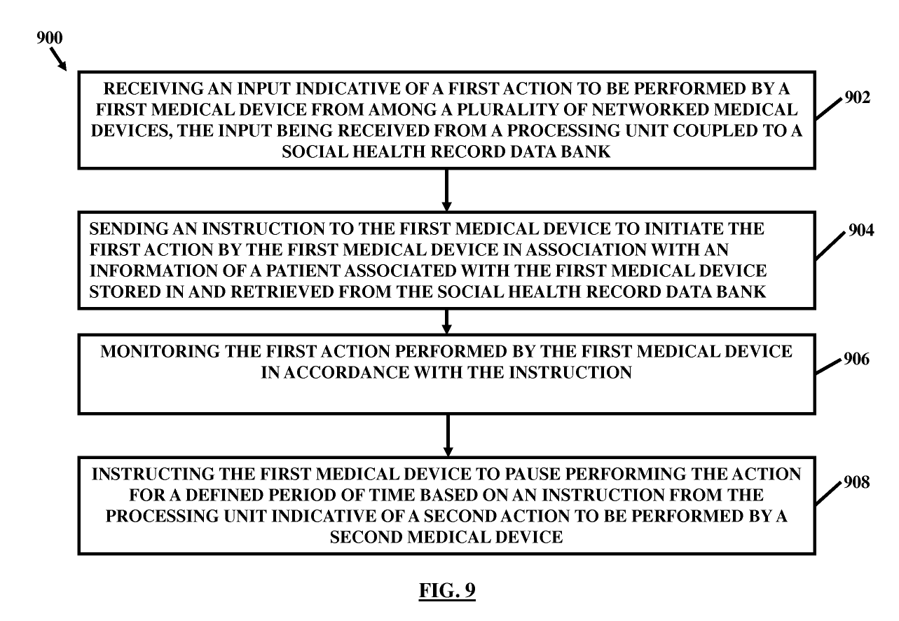

FIG. 9, with reference to FIGS. 1 through 8, illustrates a method 900 for facilitating coordinated functioning of the plurality of medical devices over the network 104. At step 902, the method 900 includes receiving the input indicative of the first action to be performed by the first medical device D1 from among the plurality of networked medical devices 402. The input is received from the server 404 coupled to the SHRDB 108. At step 904, the method 900 includes sending the instruction to the first medical device D1 to initiate the first action by the first medical device D1 in association with the information of the patient 406 associated with the first medical device D1 stored in and retrieved from the SHRDB 108. At step 906, the method 900 includes monitoring the first task performed by the first medical device D1 in accordance with the instruction. At step 908, the method 900 includes instructing the first medical device D1 to pause performing the first task for the defined period of time based on the instruction from the server 404 indicative of the second action to be performed by a second medical device D2.

In an embodiment, the method 900 may further include monitoring the defined period of time during which the first medical device D1 pauses performing the action, and the second medical device D2 performs the second action. The method 900 may include generating a signal upon completion of the defined period of time by the time detection circuit 506. The signal may be indicative of resuming the first action performed by the first medical device D1 and stopping of the second action performed by the second medical device D2. The method 900 may further include detecting an operating state of the second medical device D2, by the device state detection circuit 508, which defines performance or non performance of the second action by the second medical device D2 during the defined time period. The operating state of the first medical device D1 that defines performance or non performance of the first medical device D1 may be based on the operating state of the second medical device D2. The method 900 may also include determining the interdependence between the operating states or the tasks performed by the first medical device D1 and the second medical device D2. The method 900 may also include switching the operating states of the one or more of the first medical device D1 and the second medical device D2 upon receipt of the instruction from the controller circuit 504 and upon generation of the signal by the time detection circuit 506. The method 900 may also include generating a signal indicative of a fault by the fault detection circuit 514 if the device state detection circuit 508 does not detect a change in the operating states of either of the first and second medical devices D1, D2 upon receipt of the instruction from the controller circuit 504 to initiate or pause the first action and upon generation of the signal by the time detection circuit 506 to stop the second action and resume the first action on completion of the defined time.

In an embodiment, the method 900 may employ the plurality of medical devices 402 including even more than two medical devices. The method 900 may include in such cases determining a sequence of operations performed by the plurality of medical devices 402. The sequence of operations may depend on the interdependence of the tasks performed by the plurality of the medical devices 402. In cases of conflict between at least any two tasks, the method 900 may also include determining a priority task to be performed over other interdependent tasks. The priority may be determined based on various parameters as discussed above.

In an embodiment, the method 900 may include identifying measures or values of physiological characteristics of the patient 406 before switching an operating state of a medical device D1, D2. The method 900 may further include identifying measures or values of physiological characteristics of the patient 406 after switching the operating state of the medical device D1, D2. The method 900 may further include determining a change in the measures of the physiological characteristics and mapping the measures with the threshold values of the physiological characteristics under defined conditions. The method 900 may include rejection switching of the operating state upon determining the change as exceeding the threshold level of the physiological characteristics. For example, if the switching involves putting a ventilator in an off state for thirty minutes, the method 900 may determine the change either after switching for a short time or forecasting an affect of putting the ventilator in the off state without actually putting it off. If the change is unacceptable for example of the heart beat goes extremely down below the threshold level, the request for switching the ventilator in the off state may be rejected.

It must be appreciated that various embodiments discussed in conjunction with FIGS. 1-3 may be combined with various embodiments implementing the device integration, device coordination, and device control functionalities as disclosed in conjunction with FIGS. 4-9 to implement the data of the devices 402 in the SHRDB 108 in real time.

The embodiments herein may be embodied as a computer program product configured to include a pre-configured set of instructions, which when performed, can result in actions as stated in conjunction with the methods described above. In an example, the pre-configured set of instructions can be stored on a tangible non-transitory computer readable medium or a program storage device. In an example, the tangible non-transitory computer readable medium can be configured to include the set of instructions, which when performed by a device, can cause the device to perform acts similar to the ones described here. Embodiments herein may also include tangible and/or non-transitory computer-readable storage media for carrying or having computer executable instructions or data structures stored thereon. Such non-transitory computer readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer executable instructions, associated data structures, and program modules represent examples of the program code means for executing steps of the methods disclosed herein. The particular sequence of such executable instructions or associated data structures represents examples of corresponding acts for implementing the functions described in such steps.

The techniques provided by the embodiments herein may be implemented on an integrated circuit chip (not shown). The chip design is created in a graphical computer programming language, and stored in a computer storage medium (such as a disk, tape, physical hard drive, or virtual hard drive such as in a storage access network). If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly. The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips can be distributed by the fabricator in raw wafer form (that is, as a single wafer that has multiple unpackaged chips), as a bare die, or in a packaged form. In the latter case the chip is mounted in a single chip package (such as a plastic carrier, with leads that are affixed to a motherboard or other higher level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

The embodiments herein can include both hardware and software elements. The embodiments that are implemented in software include but are not limited to, firmware, resident software, microcode, etc.

Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

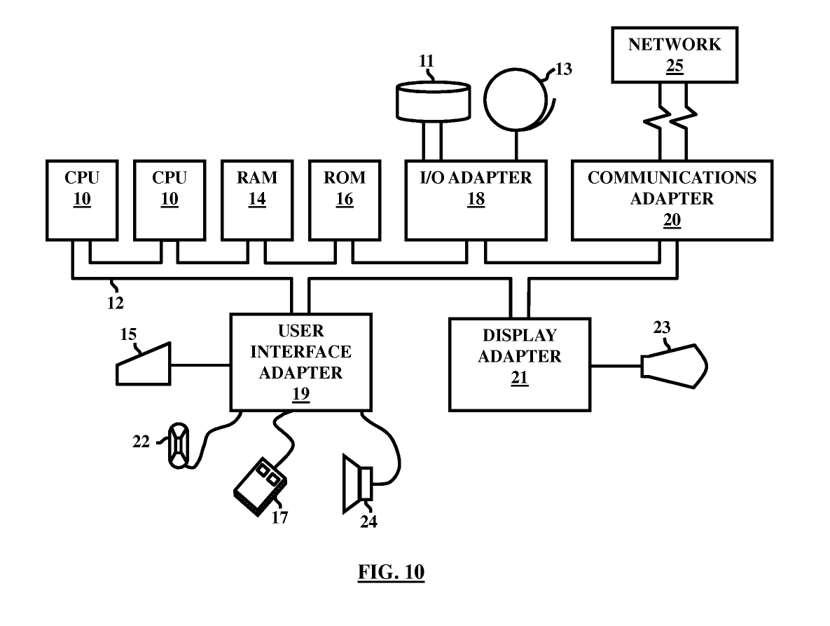

A representative hardware environment for practicing the embodiments herein is depicted in FIG. 10, with reference to FIGS. 1 through 9. This schematic drawing illustrates a hardware configuration of an information handling/computer system in accordance with the embodiments herein. The system comprises at least one processor or central processing unit (CPU) 10. The CPUs 10 are interconnected via system bus 12 to various devices such as a random access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system can read the inventive instructions on the program storage devices and follow these instructions to execute the methodology of the embodiments herein. The system further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network 25, and a display adapter 21 connects the bus 12 to a display device 23 which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A system for facilitating coordinated functioning of medical devices over a network, said system comprising:
a communication circuit configured to:
receive an input indicative of an action to be performed by a first medical device from among a plurality of networked medical devices, said input being received from a processing unit coupled to a social health record data bank (SHRDB); and
send an instruction to said first medical device to initiate said action by said first medical device in association with information of a patient associated with said first medical device stored in and retrieved from said social health record data bank; and
a control circuit configured to monitor said action performed by said first medical device and instruct said first medical device to pause performing said action for a defined period of time based on an instruction from said processing unit indicative of an action to be performed by a second medical device.

2. The system of claim 1, wherein said control circuit comprises a time detection circuit configured to monitor said defined period of time during which said first medical device pauses performing said action, and said second medical device performs a second action, wherein said time detection circuit generates a signal upon completion of said defined period of time, said signal being indicative of resuming said first action performed by said first medical device and stopping of said second action performed by said second medical device.

3. The system of claim 2, wherein said control circuit further comprises a device state detection circuit coupled to said time detection circuit and configured to identify an operating state of said second medical device which defines performance or non performance of said second action by said second medical device during said defined time period, such that an operating state of said first medical device that defines performance or non performance of said first medical device is based on said operating state of said second medical device.

4. The system of claim 3, further comprising a switch matrix coupled to each of said first and second medical devices and configured to cause switching of said operating states of said one or more of said first medical device and said second medical device upon receipt of said instruction from said controller circuit and upon generation of said signal by said time detection circuit.

5. The system of claim 4, further comprising a frequency detector configured to determine a frequency of switching of said operating state of said first medical device from a performance state to a non-performance state of said first action.

6. The system of claim 5, wherein said frequency counter circuit is configured to:
map said current frequency of switching with a threshold frequency established by said SHRDB based on a set of physiological parameters in association with said information associated with said patient; and
reject said switching of said operating state of said first medical device if said current frequency of switching equals said threshold frequency.

7. The system of claim 6, further comprising a fault detection circuit configured to generate a signal indicative of a fault if said device state detection circuit does not detect a change in said operating states of either of said first and second medical devices upon receipt of said instruction from said controller circuit to initiate or pause said first action and upon generation of said signal by said time detection circuit to stop said second action and resume said first action on completion of said defined time.

8. The system of claim 1, wherein said first medical device is authorized to reject to pause performing said first action based on said instruction received from said control circuit, wherein said rejection by said first medical device influences said section action to be performed by said second medical device, such that said SHRDB is informed about said rejection to determine an alternative action for said second medical device or an alternative medical device.

9. The system of claim 1, wherein said first medical device comprises a ventilator and second medical device comprises an X-ray machine such that said ventilator is configured to perform said first action upon receipt of said instruction to perform said first action from said communication circuit, and pause from performing of said first action upon receipt of said instruction to pause said first action from said control circuit when said control circuit instructs said X-ray machine to initiate performing said second action.

10. The system of claim 1, further comprising a physiological sensor configured to be coupled to said patient to identify a set of physiological characteristics of said patient during performance and non-performance of said first and said second medical devices, said physiological characteristics being one of a blood pressure, heart beat, respiratory rate, body temperature, and glucose level.

11. The system of claim 10, wherein said performance or non performance of said first and second medical device based on said instruction received from said processing unit of said SHRDB is rejected upon indication of a change in said physiological characteristics beyond respective threshold values corresponding to each of said physiological characteristics.

12. The system of claim 1, wherein said threshold values are dynamically calculated by said SHRDB based on age, disease, gender, and height-weight index of said patient and are communicated to said system for storage locally within said system in real time.

13. The system of claim 1, further comprising a memory circuit to store said information received from said processing unit coupled to said server and patient-specific information obtained from said SHRDB, the memory circuit further configured to store a set of threshold values of physiological parameters associated with a patient and obtained from said SHRDB.

14. A method for facilitating coordinated functioning of a plurality of medical devices over a network, said method comprising:
receiving an input indicative of a first action to be performed by a first medical device from among a plurality of networked medical devices, said input being received from a processing unit coupled to a social health record data bank (SHRDB);
sending an instruction to said first medical device to initiate said first action by said first medical device in association with information of a patient associated with said first medical device stored in and retrieved from said social health record data bank;
monitoring said first action performed by said first medical device in accordance with said instruction; and
instructing said first medical device to pause performing said action for a defined period of time based on an instruction from said processing unit indicative of a second action to be performed by a second medical device.

15. The method of claim 14, further comprising monitoring said defined period of time during which said first medical device pauses performing said action, and said second medical device performs said second action, wherein a signal is generated upon completion of said defined period of time by a time detection circuit, said signal being indicative of resuming said first action performed by said first medical device and stopping of said second action performed by said second medical device.

16. The method of claim 15 further comprising detecting an operating state of said second medical device, by a device state detection circuit, which defines performance or non performance of said second action by said second medical device during said defined time period, such that an operating state of said first medical device that defines performance or non performance of said first medical device is based on said operating state of said second medical device.

17. The method of claim 16, further comprising switching the operating states of said one or more of said first medical device and said second medical device upon receipt of said instruction from said controller circuit and upon generation of said signal by said time detection circuit.

18. The method of claim 17, further comprising generating a signal indicative of a fault by a fault detection circuit if said device state detection circuit does not detect a change in said operating states of either of said first and second medical devices upon receipt of said instruction from said controller circuit to initiate or pause said first action and upon generation of said signal by said time detection circuit to stop said second action and resume said first action on completion of said defined time.

19. A program storage device readable by computer, and comprising a program of instructions executable by said computer to perform a method for facilitating coordinated functioning of a plurality of medical devices over a network, said method comprising:
receiving an input indicative of a first action to be performed by a first medical device from among a plurality of networked medical devices, said input being received from a processing unit coupled to a social health record data bank (SHRDB);
sending an instruction to said first medical device to initiate said first action by said first medical device in association with information of a patient associated with said first medical device stored in and retrieved from said social health record data bank;
monitoring said first action performed by said first medical device in accordance with said instruction; and
instructing said first medical device to pause performing said action for a defined period of time based on an instruction from said processing unit indicative of a second action to be performed by a second medical device.

20. The program storage device of claim 19, wherein said method further comprises monitoring said defined period of time during which said first medical device pauses performing said action, and said second medical device performs said second action, wherein a signal is generated upon completion of said defined period of time by a time detection circuit, said signal being indicative of resuming said first action performed by said first medical device and stopping of said second action performed by said second medical device.
